(19) United States
(12) Patent Application Publication
(10) Pub. No.: US 2019/0150829 A1
(43) Pub. Date: May 23, 2019

(54) SYSTEMS AND METHODS FOR ON-DEMAND CPAP THERAPY

(71) Applicant: Verily Life Sciences LLC, South San Francisco, CA (US)

(72) Inventors: Brian Pepin, San Francisco, CA (US); Todd Whitehurst, Belmont, CA (US)

(73) Assignee: Verily Life Sciences LLC, South San Francisco, CA (US)

(21) Appl. No.: 16/189,301

(22) Filed: Nov. 13, 2018

Related U.S. Application Data
(60) Provisional application No. 62/587,129, filed on Nov. 16, 2017.

(51) Int. Cl. A61B 5/00; A61M 16/06; A61M 16/00; A61B 5/113

(52) U.S. Cl. CPC A61B 5/4818; A61M 16/06; A61M 16/024; A61B 5/7267; A61M 2205/3569; A61B 5/4836; A61M 16/0069; A61M 2205/3592; A61B 5/113

(57) ABSTRACT

Examples of systems and methods for on-demand CPAP therapy are described herein. One example method of providing on-demand CPAP therapy includes monitoring a physiological parameter of a wearer, the physiological parameter associated with sleep apnea, the wearer wearing an air mask connected to an air supply device; detecting a sleep apnea event based on the physiological parameter; and in response to detecting the sleep apnea event, activating the air supply device.

### CROSS-REFERENCE TO RELATED APPLICATIONS

This application claims priority to U.S. Provisional Application No. 62/587,129, filed Nov. 16, 2017, entitled “Systems and Methods for On-Demand CPAP Therapy,” which is hereby incorporated by reference in its entirety herein.

### FIELD

The present application relates to systems and methods for on-demand continuous positive air pressure (“CPAP”) therapy.

### BACKGROUND

Sleep apnea is a breathing disorder that interrupts normal breathing during sleep and that can be caused by collapse or obstruction of the airway during intakes of breath. It can affect an individual's ability to get a good night's sleep, and can trigger additional health problems, including high blood pressure, heart attacks, and strokes. Treating sleep apnea can be accomplished by providing continuous positive air pressure on an individual's airway by fitting the individual with a breathing mask connected to an air pump. The air pump then pumps air through a tube to the mask, which helps hold the individual's airway open, thereby enabling unobstructed normal breathing.

### SUMMARY

Various examples are described for systems and methods for on-demand CPAP therapy. One example method includes monitoring a physiological parameter of a wearer, the physiological parameter associated with sleep apnea, the wearer wearing an air mask connected to an air supply device; detecting a sleep apnea event based on the physiological parameter; and in response to detecting the sleep apnea event, activating the air supply device.

One example system includes an air mask; a sensor to detect a physiological parameter associated with sleep apnea; an air supply device connected to the air mask by a hollow tube, the air supply device configured to supply air pressure to the mask using the hollow tube; and a controller comprising: a non-transitory computer-readable medium; and a processor in communication with the sensor, the air supply device, and the non-transitory computer-readable medium, the processor configured to execute processor-executable instructions stored in the non-transitory computer-readable medium to: receive sensor signals from the sensor; monitor the physiological parameter based on the received sensor signals; detect a sleep apnea event based on the physiological parameter; and in response to detection of the sleep apnea event, activate the air supply device to provide the air pressure to the air mask.

One example non-transitory computer-readable medium comprising processor-executable instructions configured to cause a processor to: receive sensor signals from a sensor to detect a physiological parameter associated with sleep apnea; monitor the physiological parameter based on the received sensor signals; detect a sleep apnea event based on the physiological parameter; and in response to detection of the sleep apnea event, activate an air supply device to provide air pressure to an air mask.

These illustrative examples are mentioned not to limit or define the scope of this disclosure, but rather to provide examples to aid understanding thereof. Illustrative examples are discussed in the Detailed Description, which provides further description. Advantages offered by various examples may be further understood by examining this specification.

### BRIEF DESCRIPTION OF THE DRAWINGS

The accompanying drawings, which are incorporated into and constitute a part of this specification, illustrate one or more certain examples and, together with the description of the example, serve to explain the principles and implementations of the certain examples.

FIGS. 1-3 show example systems for on-demand CPAP therapy;

FIG. 4 shows an example method for on-demand CPAP therapy; and

FIG. 5 shows an example computing device suitable for use with example systems or methods for on-demand CPAP therapy.

### DETAILED DESCRIPTION

Examples are described herein in the context of systems and methods for on-demand CPAP therapy. Those of ordinary skill in the art will realize that the following description is illustrative only and is not intended to be in any way limiting. Reference will now be made in detail to implementations of examples as illustrated in the accompanying drawings. The same reference indicators will be used throughout the drawings and the following description to refer to the same or like items.

In the interest of clarity, not all of the routine features of the examples described herein are shown and described. It will, of course, be appreciated that in the development of any such actual implementation, numerous implementation-specific decisions must be made in order to achieve the developer's specific goals, such as compliance with application- and business-related constraints, and that these specific goals will vary from one implementation to another and from one developer to another.

To provide on-demand CPAP therapy, an individual (the “wearer”) uses an on-demand CPAP device. To use the device, the wearer puts on an air mask prior to going to sleep. The air mask is connected by an air hose or tube to a device that can supply pressurized air (the “air supply device”) to the air mask via the air hose. In this example, the air supply device is an air pump that can be activated to pump air to the air mask. However, unlike a convention CPAP therapy device that, when turned on by the wearer, supplies a continuous, constant air pressure to the wearer's air mask, when the wearer activates the example on-demand CPAP device, the air pump is not activated. Instead it remains in an inactive or standby state. The wearer can then go to sleep.

In this example, the air mask has a non-invasive blood oxygen sensor that can detect the wearer's blood oxygen saturation levels (e.g., “SvO₂” or “SpO₂”). The blood oxygen sensor is affixed to the air mask and is positioned so that it rests on the wearer's brow bone between her eyes. The blood oxygen sensor in this example shines lights into the wearer's skin and detects blood oxygen based on absorption of the light by blood vessels in the wearer's skin. A controller connected to the blood oxygen sensor obtains blood oxygen signals and determines the wearer's blood oxygen levels. It then monitors those levels over time and, if the blood oxygen level drops below a threshold, e.g., 90%, and blood oxygen level is decreasing faster than an average rate of change for the wearer (blood oxygen levels may fluctuate over time), the controller may determine a sleep apnea event is occurring or is about to occur.

After determining a sleep apnea event is occurring (or is about to), the controller activates the air supply device, which supplies pressurized air to the air mask to open the wearer's airway, thereby halting or preventing the sleep apnea event. The controller may allow the air supply device to remain active for a few seconds, e.g., up to ten seconds, before deactivating it and returning to an inactive or standby state. Thus, this example on-demand CPAP system is able to activate an air supply device only upon detection of an impending (or in-progress) sleep apnea event. Such a system may be desirable as it may eliminate some of the side effects associated with traditional CPAP machines, such as sleep difficulties due to noise from a constantly running air pump, dried out mouth and gums, rhinitis or nasal congestion, headaches, etc. Instead, by selectively activating the air supply device and deactivating it after a dose of pressurized air has been administered, the wearer may experience fewer or no side effects. In addition, because the air supply is not continuously running all night, alternate types of air supply devices may be used, such as a small cartridge of compressed air rather than a pump. Still further advantages may be obtained from reviewing this detailed description and the accompanying figures.

It should be appreciated that for purposes of this disclosure, the term “sleep apnea event” relates to a partial or total collapse or obstruction of the wearer's airway while the wearer is asleep, and may include the wearer's response to such an event, including gasping for breath, snoring, choking, etc. Thus, a “sleep apnea event” may be detected before the wearer involuntarily responds to the event by detecting physiological parameters indicating that a sleep apnea event is occurring or is likely to occur within the near future, e.g., within a few seconds. While “sleep apnea event” generally refers to a period of time before a wearer responds to a collapsed or obstructed airway, as well as the period time while the wearer is responding to such an event, the two different time periods may be separately referred to as an “impending sleep apnea event” and an “in-progress sleep apnea event.”

This illustrative example is given to introduce the reader to the general subject matter discussed herein and the disclosure is not limited to this example. The following sections describe various additional non-limiting examples and examples of systems and methods for on-demand CPAP therapy.

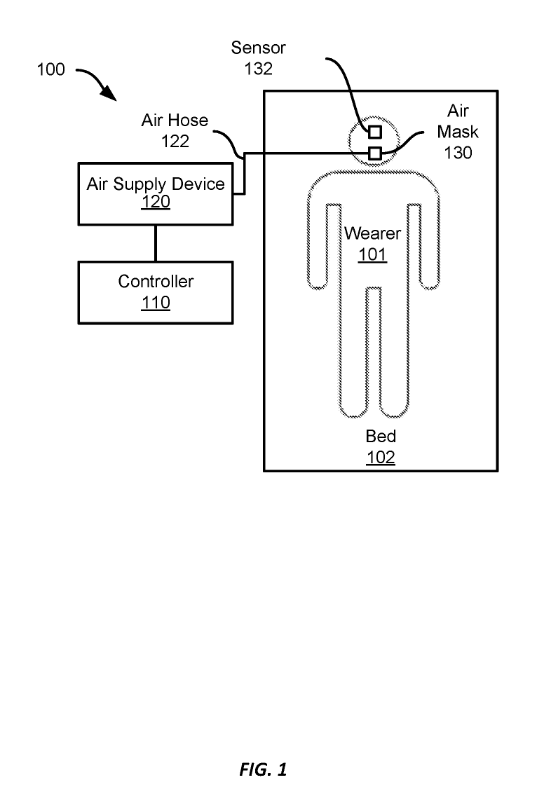

Referring now to FIG. 1, FIG. 1 shows an example on-demand CPAP system 100. The system 100 includes a controller 110, an air supply device 120, and an air mask 130, which is connected to the air supply device 120 by an air hose 122. The system 100 also includes one or more sensors 132 to sense physiological parameters of the wearer 101 sleeping on the bed 102, which will be discussed in more detail below. The sensors 132 may be incorporated into the air mask 130 or may be separately attached to the wearer in different examples.

To provide on-demand CPAP therapy, the sensors 132 transmit sensor signals to the controller 110, which uses the sensor signals to monitor the wearer for indications of a sleep apnea event. If the controller 110 detects an impending sleep apnea event, or one that is in progress, it activates the air supply device 120 for a period of time and then deactivates the air supply device 120 again. Thus, unless a detected sleep apnea event is being addressed, the controller 110 maintains the air supply device 120 in an inactive or standby state.

To enable the controller 110 to detect a sleep apnea event, the system 110 includes one or more sensors 132 that sense physiological parameters of the wearer. The physiological parameters may indicate information such as blood oxygen level, breathing rates, air volume per breath, etc. Suitable sensors may include blood oxygen sensors, accelerometers, gas flow sensors, pressure sensors, microphones, cameras, etc. Such sensors may be coupled to the air mask 130, may be worn by the wearer 101, or may be located within the room with the wearer 101. For example a microphone may be located on the wearer's nightstand or air mask 130 to measure breathing sounds, or a camera may be mounted to the bed to monitor the wearer's chest movements. The sensors 132 may be powered by a battery in the air mask 130, via a power wire running from the air supply device 120 (or controller 110) along the air hose 122, via wireless power transfer from another device, or any other suitable means for powering the sensor(s) 132.

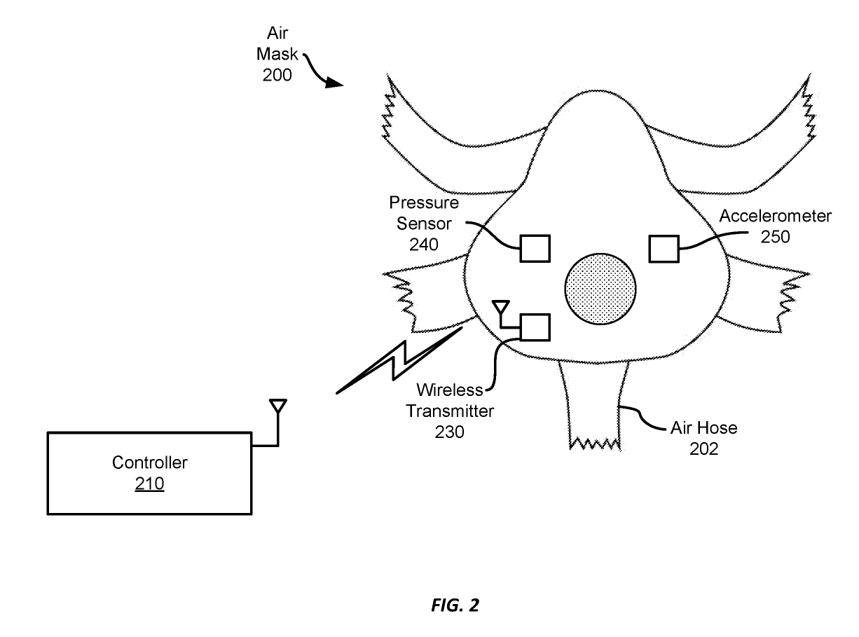

Referring now to FIG. 2, FIG. 2 shows an example arrangement of sensors 240-250 on an air mask 200 that may be employed according to some examples of on-demand CPAP systems and methods. In this example, two sensors 240-250 are attached to the air mask 200—a pressure sensor 210 and an accelerometer 220. The pressure sensor 210 obtains information relating to gas pressure within the air mask 200 that may be used to determine a breathing rate or how hard the wearer is breathing, e.g., shallow or deep breaths. The accelerometer 220 may detect the wearer's movements, such as head movement, mouth movement, etc. Such movements may be associated with breathing difficulties that indicate a sleep apnea event.

The two sensors 240-250 are coupled to a wireless transmitter 230 that is used to transmit sensor signals to the controller 210. The controller 210 may then use the received sensor signals to determine sleep apnea events.

While the example shown in FIG. 2 employs a pressure sensor 240 and an accelerometer 250, any suitable number or type of sensors, such as those discussed above, may be employed according to different examples.

Referring again to FIG. 1, the sensor 132 in this example communicates wirelessly with the controller 110 using a Bluetooth (“BT”) low energy (“BLE”) communication technique, however, any suitable wired or wireless communications technique may be employed, including BT, WiFi, ZigBee, near-field communications (“NFC”), Ethernet, serial protocols (e.g., RS-232, RS-485, etc.), parallel communication techniques, etc. In some examples, the sensor 132 may be provide an analog voltage or current signal.

In this example, the controller 110 is a computing device that is separate and distinct from the air supply device 120 and the air mask 130, and may reside within a housing, such as on the user's nightstand, the floor, or elsewhere within proximity of the wearer. However, in some examples, the controller 110 may be built into the air supply device 120, the air mask 130, or the air hose 122.

The controller 110 is in communication with the air supply device 120, such as using one or more of the wired or wireless techniques discussed above. For example, to activate or deactivate the air supply device 120, the controller 110 may change a logic state of a control line to the air supply device 120 to toggle between active and inactive (or standby) states, or may transmit a message to the air supply device 120 indicating parameters regarding how to activate and supply pressurized air to the air mask 130.

As discussed above, the controller 110 receives signals from one or more sensors 132 and activates or deactivates the air supply device 120 when a sleep apnea event is detected. After detecting a sleep apnea event, the controller 110 may activate the air supply device 120 and continue monitoring sensor signals from the sensor(s) 132. When the sensor signals indicate that a sleep apnea event is no longer detected, the controller 110 deactivates the air supply device 120, which returns to an inactive or standby state. The controller 110 then continues to monitor the sensor signals to detect any additional sleep apnea events.

In this example, the air supply device 120 is an air pump, but any suitable air supply device may be employed. For example, an air supply device 120 may include a container of pressurized air coupled to a nozzle or valve that can be selectively opened to provide the pressurized air to the air mask 130 via the air hose 122. Still other suitable means for providing pressurized air to the air mask 130 may be employed according to different examples.

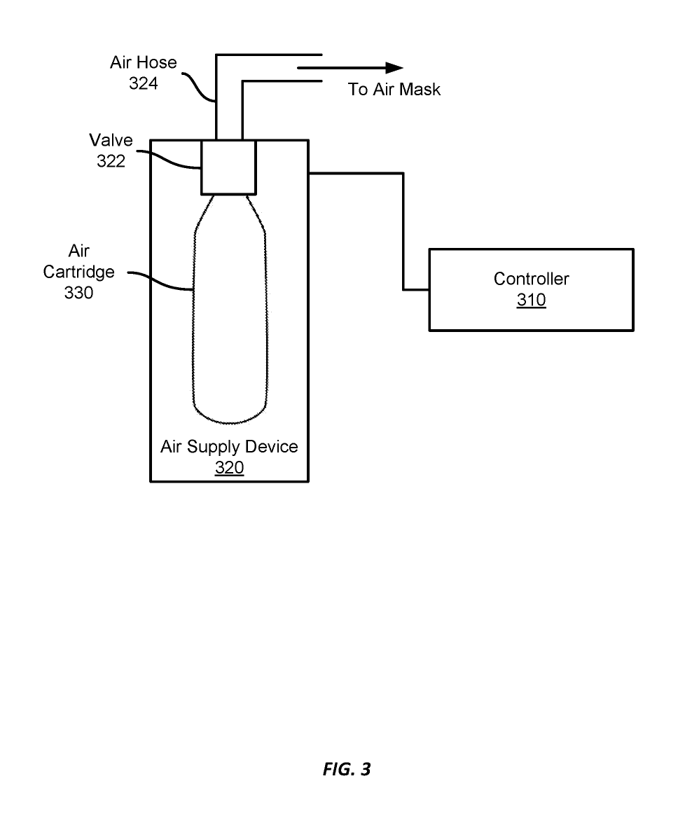

Referring now to FIG. 3, shows an example air supply device 320 in communication with a controller 310 as a part of an on-demand CPAP system. The air supply device 320 in this example has a housing in which is an air cartridge 330 having pressurized air within it, e.g., stored at 300 psi prior to the first use. The air cartridge 330 is connected to a valve 322 that can be opened to allow a quantity of pressurized air to exit the air cartridge 330 and traverse the air hose 324 to an air mask. In this example, the valve 322 also includes a pressure regulator that regulates the pressure of the expelled pressurized air to maintain a safe pressure to send to the air mask. For example, the pressure may be regulated to a range between 4 and 30 centimeters of water (cmH₂O) inclusive, pressure units frequently used in CPAP systems.

The valve 322 may be actuated by a signal from the controller 310 based on the configured air pressure for the system, e.g., 15 cmH₂O. In this example, the valve 322 is selected based on the maximum pressure it will provide to the air hose 324, such as 30 cmH₂O, and the controller 310 may toggle the valve between fully open and fully closed states. Thus, the air supply device 320 may supply 30 cmH₂O every time it is activated (provided sufficient air pressure remains within the air cartridge). In some examples, however, the controller 310 may output an analog or pulse-width modulated signal to the valve 322 to partially open it to provide a determined air pressure to the air mask. For example, if the controller 310 determines an impending sleep apnea event, it may output a signal to open the valve 322 to allow 10 cmH₂O of air pressure into the air mask. But if the controller determines an in-progress sleep apnea event, it may output a signal to open the valve to allow 20 cmH₂O of air pressure into the air mask. To deactivate the air supply device, the controller 310 may output a signal to the air supply device 320 to close the valve 322.

In some examples, the controller 310 may receive information from the air supply device 320. For example, the valve 322 may include a pressure sensor that can provide information indicating the air pressure within the air cartridge 330. The controller 310 can receive this information from the air supply device 320, and if it determines that the air pressure within the cartridge 330 is too low, or if the air supply device 320 provides a signal indicating low air pressure, the controller 310 can output a notification to the wearer indicating that the air cartridge 33 needs to be refilled or replaced.

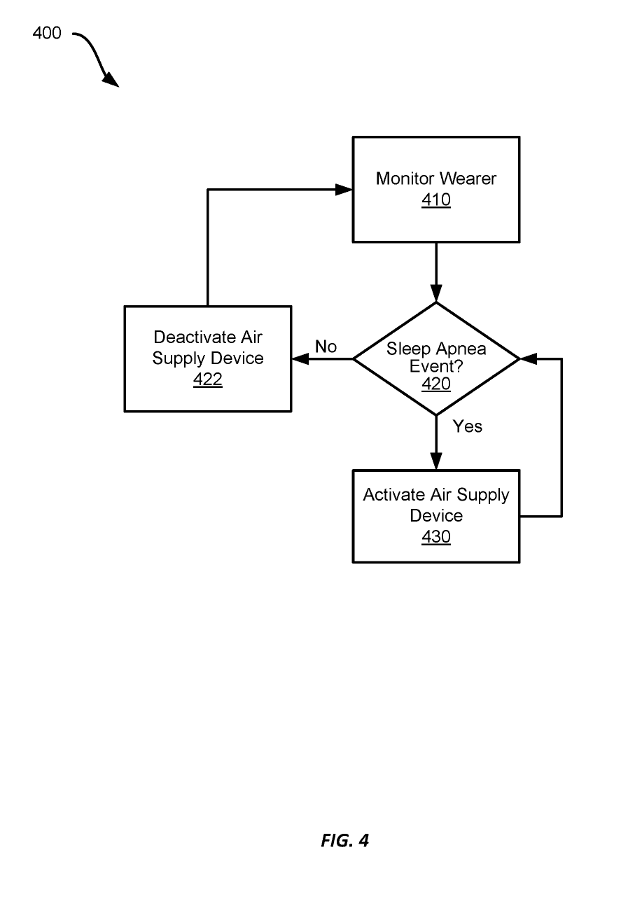

Referring now to FIG. 4, FIG. 4 shows an example method 400 for on-demand CPAP therapy. This example method 400 will be described with respect to the example system 100 shown in FIG. 1, but it should be appreciated that the method could be performed with any suitable system according to this disclosure.

At block 410, the controller 410 monitors a physiological parameter of a wearer 101. In this example, the wearer 101 is wearing an air mask 130 that includes a blood oxygen sensor 132. The blood oxygen sensor 132 obtains blood oxygen measurements and provides them to the controller 110 using a wireless communication technique.

In this example, the controller 110 monitors the measured blood oxygen level against a predetermined blood oxygen threshold. In this example, the blood oxygen threshold is 90%, though any suitable threshold may be selected. For example, the controller 110 may determine the wearer's average minimum blood oxygen level during normal breathing and establish a threshold that is five percentage points below, or, e.g., 95% of, the determined average. Thus, if the wearer has an average minimum blood oxygen level of 94%, the controller may establish a threshold of 89%.

In addition to monitoring the instantaneous blood oxygen level, the controller 110 may also monitor a rate of change in the blood oxygen level. During normal breathing, the wearer's blood oxygen level may fluctuate above and below an average blood oxygen level for the wearer. For example, shortly after an intake of breath, the blood oxygen level may steadily rise to 99%, then steadily decrease to 94% prior to inhale of the next breath. Thus, the wearer's blood oxygen level may fluctuate between 94% and 99% during normal breathing, with an average of approximately 97%. The controller 110 may determine an average rate of change of the blood oxygen level as it decreases from 99% to 94%.

For example, the controller may determine a slope or gradient between measurements as the wearer's blood oxygen levels decrease from 99% to 94% (or from a measured maximum to a measured minimum blood oxygen level). Similarly a slope or gradient may be determined between measurements as the wearer's blood oxygen level increases from a measured minimum to a measured maximum blood oxygen level. The controller 110 may then accumulate these slopes or gradients over successive breathing cycles and average them to determine an average rate of change, which may differ for decreasing blood oxygen levels and increasing blood oxygen levels. In some examples, the controller 110 may calculate a running average over a predetermined number of breathing cycles (e.g., ten breathing cycles), or it may periodically recalculate the averages periodically, such as every ten minutes.

While this example has discussed the use of a blood oxygen sensor, other sensors may be employed instead, or in addition. For example, the system 100 may employ one or more accelerometers in the air mask to detect head movement or mouth movement. The accelerometers may provide acceleration information to the controller in one or more degrees of freedom. The accelerometer data may then be used to determine the user's average movements, or the information may be compared, e.g., using a trained machine learning model, against data associated with sleep apnea events, such as sudden head movements, gasping, coughing, etc. The machine learning model may provide outputs indicating whether abnormal movements were detected, and whether those abnormal movements are indicative of a sleep apnea event.

In some examples, the system 100 may employ one or more pressure sensors in the air mask 130 to detect changes in air pressure due to the wearer's breathing cycles. The changes in pressures may be rhythmic or periodic during normal breathing, but prior to or during a sleep apnea event, the pressure changes may become more erratic or may cease altogether if the wearer stops breathing. Thus, sensor information received from the pressure sensors may be monitored by the controller 110 to detect potential sleep apnea events. For example, a machine learning model may be trained based on the wearer's typical breathing cycle and may be able to detect and output notifications if the breathing cycle is interrupted.

At block 420, the controller 110 determines whether a sleep apnea event has been detected based on the monitored physiological parameter and one or more conditions within a set of conditions. In this example, the controller 110 monitors blood oxygen levels as discussed above and evaluates both instantaneous blood oxygen levels and rates of change of blood oxygen levels against the conditions within the set of conditions. In this example, the conditions include the following:
(i) instantaneous blood oxygen level less than 92%?
(ii) rate of change of blood oxygen level greater than 110% of average rate of change?
(iii) decreasing blood oxygen level?

The controller 110 may then determine whether one or more of the conditions are satisfied to determine whether a sleep apnea event has been detected. In this example, the controller 110 may detect a sleep apnea event if conditions (i) and (ii) are true, or if conditions (i) and (iii) are true, or if all three conditions are true.

In this example, the controller 110 monitors both instantaneous blood oxygen levels and rates of change of blood oxygen levels over a rolling time period of approximately ten seconds. Thus, the controller 110 may evaluate condition (i) with respect to the instantaneous blood oxygen level based on a most recent sensor signal, while it may evaluate conditions (ii) and (iii) using a determined rate of change based on two or more of the most recent sensor signals. Such conditions may be used to detect that the user's blood oxygen levels are lower than usual, indicating a potential breathing issue. Further, a higher-than-average rate of decrease may indicate that the breathing cycle has been disturbed in some way, and a lower-than-average blood oxygen level that continues to decrease may further indicate breathing difficulty. Thus, such conditions may indicate that a sleep apnea event is occurring.

As discussed above, other types of sensors may be employed instead of, or in addition to, blood oxygen sensors. The controller 110 may employ conditions for sensor signals from other types of sensors. For example, sensor signals from one or more accelerometers may be analyzed to determine head or mouth movement. The controller 110 may employ a machine learning technique to determine different types of head or mouth movements based on the received sensor signals. The determined head or mouth movements may be used to determine whether one or more conditions have been satisfied, such as (i) is the head movement a jerking movement?, (ii) is the head movement a rolling movement?, (iii), is the head movement a rocking movement?, (iv) is the mouth opening quickly?, etc. One or more of these conditions may provide an indication that a sleep apnea event is occurring.

In addition, one or more pressure sensors (or air flow sensors) may be affixed to the air mask 130 to detect air changes in air pressure within the air mask 130. During normal breathing, pressure changes (or changes in air flow) may be cyclical and regular. But during a sleep apnea event, changes may be less pronounced, a cyclical pattern may change in frequency or amplitude, or may halt entirely. Thus, conditions associated with deviations from a cycle of pressure or air flow changes may be employed to detect potential sleep apnea events.

Additional types of sensors may be employed, such as cameras or microphones may be employed to detect sleep apnea events. Microphones may record audio signals of the wearer's breathing cycle, and may be used to detect deviations from a regular breathing cycle or if the wearer stops breathing for a period of time. A camera may be used to monitor chest movement and the controller 110 may detect sleep apnea events if monitored chest movements deviate from a regular cycle, or stop entirely for a period of time. Still additional types of sensors may be used according to different examples to detect deviations from a wearer's normal breathing cycle.

While the above examples include examples employing different types of sensors, conditions for multiple different sensor types may be combined to obtain multiple different indicators to determine whether a sleep apnea event is occurring. The fusion of these different types of sensors and their respective sensor information may provide a more robust system for early detection of sleep apnea events, including detecting the events before they have interrupted the wearer's sleep cycle, e.g., before the user begins gasping for breath or wakes up.

At block 422, the controller 110 deactivates the air supply device 120. For example, the controller 110 may entirely deactivate the air supply device 120, such as by disconnecting power to an air pump, or closing a valve from a pressurized air cartridge. In some examples, the controller 110 may put the air supply device 120 in a standby mode. For example, the controller 110 may leave an air pump running, but close a vent to divert air from the air hose 122 into the environment. In such an example, the controller 110 may be able to more quickly reactivate the air pump by changing a valve position rather than reactivating the air pump, which may involve a delayed response as the pump must return to an operating speed.

At block 430, the controller 110 activates the air supply device 120. In this example, the controller 110 transmits a signal to the air supply device 120 to turn on an air pump. The air pump turns on and spins up to an operating speed over a short period of time (e.g., a few seconds), thereby pumping air through the air hose 122 into the air mask 130. In another example, the controller 110 closes a valve in the air supply device 120, where the air pump remains active at all times. By closing the valve, the controller 110 redirects air from being vented into the wearer's room and into the air hose 122 and to the air mask 130. In a further example, the controller 110 opens a valve coupled to a pressurized air cartridge to vent air from the cartridge to the air mask 130.

In this embodiment, which describes a “closed-loop” on-demand CPAP method, after the air supply device has been activated at block 430, the method returns to block 420, where the controller continues to determine whether sleep apnea event persists. In some examples, however, the method 400 may be an “open-loop” on-demand CPAP method, and the method 400 may return to block 422 after a predetermined amount of time. For example, the controller 110 may deactivate the air supply device 422 after ten seconds, before returning to monitor the wearer 101 at block 410. And while certain examples have been described above, still further examples within the scope of the present disclosure.

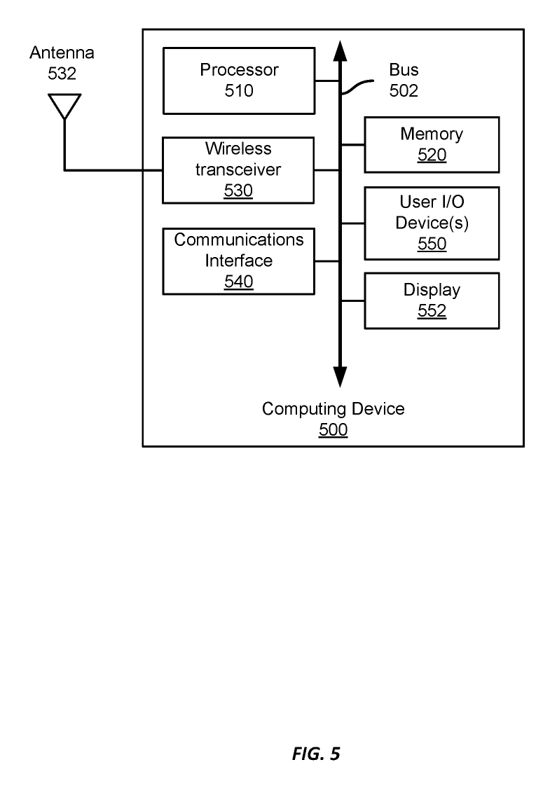

Referring now to FIG. 5, FIG. 5 shows an example computing device 500 suitable for use with one or more systems and methods for on-demand CPAP therapy according to this disclosure. The example computing device 500 includes a processor 510 which is in communication with the memory 520 and other components of the computing device 500 using one or more communications buses 502. The processor 510 is configured to execute processor-executable instructions stored in the memory 520 to perform insect sensing according to different examples, such as part or all of the example method 400 described above with respect to FIG. 4. The computing device, in this example, also includes one or more user input devices 550, such as a keyboard, mouse, touchscreen, microphone, etc., to accept user input. The computing device 500 also includes a 552 display to provide visual output to a user.

The computing device also 500 includes a wireless transceiver 530 and corresponding antenna 532 to allow the computing device 500 to communicate wirelessly using any suitable wireless communication protocol, including WiFi, Bluetooth (“BT”), cellular, etc. techniques. The computing device 500 also includes a communications interface 540 that enables communications with external devices, such as one or more sensors, as described above. In some examples, the communications interface 540 may enable communications using one or more networks, including a local area network (“LAN”); wide area network (“WAN”), such as the Internet; metropolitan area network (“MAN”); point-to-point or peer-to-peer connection; etc. Communication with other devices may be accomplished using any suitable networking protocol. For example, one suitable networking protocol may include the Internet Protocol (“IP”), Transmission Control Protocol (“TCP”), User Datagram Protocol (“UDP”), or combinations thereof, such as TCP/IP or UDP/IP.

While some examples of methods and systems herein are described in terms of software executing on various machines, the methods and systems may also be implemented as specifically-configured hardware, such as field-programmable gate array (FPGA) specifically to execute the various methods. For example, examples can be implemented in digital electronic circuitry, or in computer hardware, firmware, software, or in a combination thereof. In one example, a device may include a processor or processors. The processor comprises a computer-readable medium, such as a random access memory (RAM) coupled to the processor. The processor executes computer-executable program instructions stored in memory, such as executing one or more computer programs. Such processors may comprise a microprocessor, a digital signal processor (DSP), an application-specific integrated circuit (ASIC), field programmable gate arrays (FPGAs), and state machines. Such processors may further comprise programmable electronic devices such as PLCs, programmable interrupt controllers (PICs), programmable logic devices (PLDs), programmable read-only memories (PROMs), electronically programmable read-only memories (EPROMs or EEPROMs), or other similar devices.

Such processors may comprise, or may be in communication with, media, for example computer-readable storage media, that may store instructions that, when executed by the processor, can cause the processor to perform the steps described herein as carried out, or assisted, by a processor. Examples of computer-readable media may include, but are not limited to, an electronic, optical, magnetic, or other storage device capable of providing a processor, such as the processor in a web server, with computer-readable instructions. Other examples of media comprise, but are not limited to, a floppy disk, CD-ROM, magnetic disk, memory chip, ROM, RAM, ASIC, configured processor, all optical media, all magnetic tape or other magnetic media, or any other medium from which a computer processor can read. The processor, and the processing, described may be in one or more structures, and may be dispersed through one or more structures. The processor may comprise code for carrying out one or more of the methods (or parts of methods) described herein.

The foregoing description of some examples has been presented only for the purpose of illustration and description and is not intended to be exhaustive or to limit the disclosure to the precise forms disclosed. Numerous modifications and adaptations thereof will be apparent to those skilled in the art without departing from the spirit and scope of the disclosure.

Reference herein to an example or implementation means that a particular feature, structure, operation, or other characteristic described in connection with the example may be included in at least one implementation of the disclosure. The disclosure is not restricted to the particular examples or implementations described as such. The appearance of the phrases “in one example,” “in an example,” “in one implementation,” or “in an implementation,” or variations of the same in various places in the specification does not necessarily refer to the same example or implementation. Any particular feature, structure, operation, or other characteristic described in this specification in relation to one example or implementation may be combined with other features, structures, operations, or other characteristics described in respect of any other example or implementation.

Use herein of the word “or” is intended to cover inclusive and exclusive OR conditions. In other words, A or B or C includes any or all of the following alternative combinations as appropriate for a particular usage: A alone; B alone; C alone; A and B only; A and C only; B and C only; and A and B and C.

That which is claimed is:

1. A method comprising:
monitoring a physiological parameter of a wearer, the physiological parameter associated with sleep apnea, the wearer wearing an air mask connected to an air supply device;
detecting a sleep apnea event based on the physiological parameter; and
in response to detecting the sleep apnea event, activating the air supply device.

2. The method of claim 1, further comprising maintaining the air supply device in an inactive state during the monitoring and prior to detection of the sleep apnea event.

3. The method of claim 1, further comprising:
in response to detecting an end of the sleep apnea event, deactivating the air supply device.

4. The method of claim 1, wherein the air supply device comprises one of an air pump or a pressurized air canister.

5. The method of claim 1, wherein activating the air supply device comprises providing air pressure to an air mask worn by the wearer, wherein the air pressure comprises substantially 4-30 centimeters of water pressure.

6. The method of claim 1, wherein the physiological parameter is a blood oxygen saturation parameter.

7. The method of claim 6, wherein detecting the sleep apnea event comprises detecting the blood oxygen saturation below a predetermined threshold.

8. The method of claim 6, wherein detecting the sleep apnea event is based on a rate of change of the blood oxygen saturation for the wearer.

9. The method of claim 1, further comprising detecting a change in air pressure in an air mask, and wherein the physiological parameter is the air pressure.

10. The method of claim 1, wherein the physiological parameter comprises head movement, and wherein monitoring the head movement comprises obtaining data from an accelerometer affixed to an air mask.

11. A device comprising:
an air mask;
a sensor to detect a physiological parameter associated with sleep apnea;
an air supply device connected to the air mask by a hollow tube, the air supply device configured to supply air pressure to the mask using the hollow tube; and
a controller comprising:
a non-transitory computer-readable medium; and
a processor in communication with the sensor, the air supply device, and the non-transitory computer-readable medium, the processor configured to execute processor-executable instructions stored in the non-transitory computer-readable medium to:
receive sensor signals from the sensor;
monitor the physiological parameter based on the received sensor signals;
detect a sleep apnea event based on the physiological parameter; and
in response to detection of the sleep apnea event, activate the air supply device to provide the air pressure to the air mask.

12. The device of claim 11, wherein the processor is configured to execute processor-executable instructions stored in the non-transitory computer-readable medium to maintain the air supply device in an inactive state during the monitoring and prior to detection of the sleep apnea event.

13. The device of claim 11, wherein the sensor is a blood oxygen saturation sensor.

14. The device of claim 13, wherein the blood oxygen saturation sensor is configured to be worn on the wearer's face on a ridge of bone substantially between the wearer's eyes.

15. The device of claim 13, wherein the processor is configured to execute processor-executable instructions stored in the non-transitory computer-readable medium to detect a sleep apnea event if a blood oxygen saturation is below a predetermined threshold.

16. The device of claim 13, wherein the processor configured to execute processor-executable instructions stored in the non-transitory computer-readable medium to determine a rate of change in the blood oxygen saturation, and detect a sleep apnea event based on the rate of change in the blood oxygen saturation.

17. The device of claim 11, wherein the sensor is a pressure sensor or an accelerometer coupled to the air mask.

18. The device of claim 17, wherein the processor configured to execute processor-executable instructions stored in the non-transitory computer-readable medium to detect a change in air pressure in an air mask, and wherein the physiological parameter is the change in air pressure.

19. The device of claim 17, wherein the processor configured to execute processor-executable instructions stored in the non-transitory computer-readable medium to detect head movement based on sensor signals received from the accelerometer, and wherein the physiological parameter is head movement.

20. The device of claim 11, wherein the processor configured to execute processor-executable instructions stored in the non-transitory computer-readable medium to, in response to detection of an end of the sleep apnea event, deactivate the air supply device.

21. The device of claim 11, wherein the air supply device comprises one of an air pump or a pressurized air canister.

22. The device of claim 11, wherein the air pressure comprises substantially 4-30 centimeters of water pressure.

23. The device of claim 11, wherein the controller is coupled to the air mask.

24. A non-transitory computer-readable medium comprising processor-executable instructions configured to cause a processor to:
receive sensor signals from a sensor to detect a physiological parameter associated with sleep apnea;
monitor the physiological parameter based on the received sensor signals;
detect a sleep apnea event based on the physiological parameter; and
in response to detection of the sleep apnea event, activate an air supply device to provide air pressure to an air mask.

25. The non-transitory computer-readable medium of claim 24, wherein the processor-executable instructions are further configured to cause the processor to maintain the air supply device in an inactive state during the monitoring and prior to detection of the sleep apnea event.

26. The non-transitory computer-readable medium of claim 24, wherein the sensor is a blood oxygen saturation sensor and wherein the processor-executable instructions are further configured to cause the processor to detect a sleep apnea event if a blood oxygen saturation is below a predetermined threshold.

27. The non-transitory computer-readable medium of claim 24, wherein the sensor is a blood oxygen saturation sensor and wherein the processor-executable instructions are further configured to cause the processor to determine a rate of change in the blood oxygen saturation, and detect a sleep apnea event based on the rate of change in the blood oxygen saturation.

28. The non-transitory computer-readable medium of claim 24, wherein the sensor is a pressure sensor and wherein the processor-executable instructions are further configured to cause the processor to detect a change in air pressure in an air mask, and wherein the physiological parameter is the change in air pressure.

29. The non-transitory computer-readable medium of claim 24, wherein the sensor is an accelerometer coupled to the air mask and wherein the processor-executable instructions are further configured to cause the processor to detect head movement based on sensor signals received from the accelerometer, and wherein the physiological parameter is head movement.

30. The non-transitory computer-readable medium of claim 24, wherein the processor-executable instructions are further configured to cause the processor to, in response to detection of an end of the sleep apnea event, deactivate the air supply device.

31. The non-transitory computer-readable medium of claim 24, wherein the air supply device comprises one of an air pump or a pressurized air canister.

32. The non-transitory computer-readable medium of claim 24, wherein the air pressure comprises substantially 4-30 centimeters of water pressure.
